(19) United States
(12) Patent Application Publication
(10) Pub. No.: US 2006/0002984 A1
(43) Pub. Date: Jan. 5, 2006

(54) Diet composition and method

(76) Inventor: Adam Brook, New Haven, CT (US)

Correspondence Address: ROBERT A. PARSONS, 4000 N. CENTRAL AVENUE, SUITE 1220, PHOENIX, AZ, 85012, US

(21) Appl. No.: 11/170,870

(22) Filed: Jun. 30, 2005

Related U.S. Application Data
(60) Provisional application No. 60/584,608, filed on Jun. 30, 2004.
(60) Provisional application No. 60/602,517, filed on Aug. 17, 2004.
(60) Provisional application No. 60/609,631, filed on Sep. 13, 2004.
(60) Provisional application No. 60/654,230, filed on Feb. 18, 2005.
(60) Provisional application No. 60/663,498, filed on Mar. 16, 2005.
(60) Provisional application No. 60/681,727, filed on May 17, 2005.

(51) Int. Cl. A61K 47/00

(52) U.S. Cl. 424/439

(57) ABSTRACT

A method of dieting includes providing a first food item; determining the caloric density and the number of calories of the first food item; providing a second food item; determining the caloric density and the number of calories of the second food item; adjusting amounts of the first and second food items based on a difference between the caloric densities between them; and consuming the amounts of the first and second food items, the number of calories in the first and second food items being less than or equal to a predetermined amount of calories.

### CROSS-REFERENCE TO RELATED APPLICATIONS

This application claims the benefit of U.S. Provisional Application No. 60/584,608, filed 30 Jun. 2004; U.S. Provisional Application No. 60/602,517, filed 17 Aug. 2004; U.S. Provisional Application No. 60/609,631, filed 13 Sep. 2004; U.S. Provisional Application No. 60/654,230, filed 18 Feb. 2005; U.S. Provisional Application No. 60/663,498, filed 16 Mar. 2005; and U.S. Provisional Application No. 60/681,727, filed 17 May 2005.

### FIELD OF THE INVENTION

The present invention relates generally to diets, and more particularly, to a healthy diet for managing a person's body weight.

### BACKGROUND OF THE INVENTION

A person's choice of diet is important. A proper diet can reduce the likelihood of certain illnesses and diseases and improve the person's appearance, which may be the most common reason people diet. One disease that is directly affected by diet is obesity. The National Health and Nutrition Examination Survey estimated that 64% of U.S. adults are overweight or obese. In addition, 15% of children and adolescents are overweight or obese. The percentage of Americans who are very overweight has increased dramatically over the last 20 years, and over the last 40 years the percentage of Americans who are obese has more than doubled.

Since obesity is one of the leading health problems in America, many weight loss diets are promoted every year to assist people in gaining control over their own weight. For example, diets that promote high protein and fat intake along with low carbohydrate intake are extremely popular (See A. Agatston, The South Beach Diet, 2003 Random House, NY and R. C. Atkins, Dr. Atkins' New Diet Revolution, 2001 Avon Books NY). In one version of Dr. Atkins' diet, it is claimed that a person gains more weight from eating 1000 calories of carbohydrates than from eating 1000 calories of fat. In another example, diets that promote a reduction in fat intake are also very popular. Several studies have suggested that reducing saturated fat intake may improve blood lipid profiles and help the person lose weight in the short-term.

However, it is questionable whether or not these diets are effective in the long run. For example, several studies have found that people do not gain more weight from eating 1000 calories of carbohydrates than from eating 1000 calories of fat. Further, it has been well established in the scientific literature that a high intake of animal fats causes heart disease. Further, these diets and ones like them have never been proven to provide long-term weight loss. In fact, most people stay with these diets for a short period of time and lose weight, but then gain the weight back and often put even more weight on. This is usually because it is very difficult for people to follow these diets for long periods of time. One cause of this problem is that people generally want to eat a wide variety of foods and these diets are restrictive in the variety of foods that one can eat. Many prior art diets are not suitable for their intended purpose and they leave much to be desired from the standpoint of healthiness and the ability of the dieter to keep excess weight off in the long run. As a result, there is a need for an improved diet.

### BRIEF SUMMARY OF THE INVENTION

The present invention provides a method of dieting, which includes providing a food item; determining the caloric density of the food item; and consuming an amount of the food item based on its caloric density.

The present invention also provides a method of dieting which includes providing a first food item; determining the caloric density and the number of calories of the first food item; providing a second food item; determining the caloric density and the number of calories of the second food item; adjusting amounts of the first and second food items is based on a difference between the caloric densities between them; and consuming the amounts of the first and second food items, the number of calories in the first and second food items being less than or equal to a predetermined amount of calories.

The present invention further provides a method of dieting which includes determining a desired number of calories to consume; providing a first portion of food with a first caloric density and a first number of calories; providing a second portion of food with a second caloric density and a second number of calories, the combination of the first and second number of calories being less than or equal to the desired number of calories; and adjusting the first and second portions of food so that the first portion of food is greater than the second portion of food and the first caloric density is less than the second caloric density.

These and other features, aspects, and advantages of the present invention will become better understood with reference to the following drawings, description, and claims.

### DETAILED DESCRIPTION OF THE INVENTION

The present invention provides a diet which allows a person to lose body weight in a healthy manner. The person on this diet is more likely to keep off the lost weight and/or to maintain a healthy one. This is because the diet provides a wide variety of healthy food items which can be consumed by the person. This is advantageous because people generally like a wide variety of foods to eat. The body weight is lost in a healthy manner because the diet provides foods that are likely to reduce or do not cause disease and illness, such as heart disease and cancer. For the general population following this diet, it is anticipated that it will reduce the risk of cancer and heart disease by approximately 40% and approximately 80%, respectively.

There are many reasons why one should lose weight or maintain a healthy one. One reason is that being overweight or obese causes or contributes to a large number of diseases, such as heart disease, certain types of cancer, diabetes, stroke, arthritis, breathing problems, and sleep apnea. The cancer can be of the esophagus, colon, rectum, breast, uterus, and kidney. Obesity can also cause high blood pressure, high blood cholesterol, gallbladder disease, and psychological disorders, such as depression. These diseases and illnesses can drastically reduce a person's quality of life and can also cause diseases that are generally treated using extensive and expensive medical and surgical equipment and procedures. Further, the life span of overweight people has been estimated to be reduced by about 10%, and the life span of obese people has been estimated to be reduced by about 25%. Thus, maintaining a healthy body weight can improve a person's health and decrease the chances of having serious disabling diseases and illnesses.

There are several ways to estimate whether or not a person has a normal body weight or is obese or overweight. One way is to determine the person's body mass index (BMI). The formula used most often to estimate a person's BMI is given by BMI=[weight (in kg)]/[height(in meters)]². A body mass index between about 19 and 24.9 is considered normal and a body mass index of about 25 or over is considered overweight. A BMI between 18 and 25 has been shown to be associated with the lowest death rate and lowest incidence of obesity-related diseases. The BMIs for people of various heights has been tabulated for convenience and are readily available. As the body mass index increases, the risk of diseases and health related issues increases too.

Other ways to estimate whether the person has a normal weight or is obese or overweight is to estimate the person's body fat percentage and/or waist circumference. This can be done by using calipers, hydrostatic immersion, bioelectric impedance, and dual energy X-ray absorptiometry. Once the person has determined his or her current body weight and desired body weight, that person can plan out how to reduce the difference between the two. Most dieters will try to lose one to two pounds per week since this is the rate recommended by most physician experts as being healthy, although some dieters can try to lose weight at a greater or lesser rate. However, it should be noted that losing weight at a rate of more than one to two pound per week can cause health problems, such as gallbladder disease and a loss of muscle mass, in some people.

In accordance with the invention, the diet promotes eating foods that are healthy. There are a number of factors that go into determining whether a food is healthy. For example, healthy foods generally have low amounts of saturated fat, and/or sodium. It is generally recommended to consume less than 20 grams of saturated fat, and less than 2500 milligrams of sodium, for a 2000 calorie per day diet. However, these are general guidelines and an individual's personal health history plays into how concerned they should be about saturated fat and sodium intake. It should be noted, however, that there are other ways to determine whether a food is healthy, but they are not discussed here for simplicity.

In accordance with the invention, the diet also promotes eating foods that have a low caloric density. This is because two foods of a given volume will make the dieter feel equally full, but if one of the foods has a lower caloric density than the other one, then it will provide the person with fewer calories. The number of calories a person consumes and burns during a given time period determines whether they gain, maintain, or lose weight. A person gains weight if he or she consumes more calories than he or she burns. A person maintains weight if he or she consumes as many calories as he or she burns. A person loses weight if he or she consumes fewer calories than he or she burns. Thus, losing weight is a matter of eating fewer calories and/or burning more calories. Burning more calories can be accomplished by exercising and by being more active.

The density of any food item can be represented as a number for the convenience of the dieter in several ways. In one way, the caloric density of a food item is the number of calories in the food item divided by its weight (Caloric Density=Calories+Weight). In another way, the true caloric density of a food item is the number of calories in the food item divided by its volume (True Caloric Density=Calories+Volume). This is referred to herein as the “true caloric density” and the volume used in this calculation is that which excludes air and gas within the food item. Similarly, the number of calories the person eats is equal to the weight of the food item consumed multiplied by the caloric density of the food (Calories=Caloric Density×Weight). The dieter can compare the caloric density number to other values to see if the food item has a high caloric density, a low caloric density, or a medium caloric density.

If the caloric density of a solid food item is less than about two, then the person will generally consume fewer calories if the person eats a portion of this food. If the caloric density is between about two and three, then the person will generally consume more calories if the person eats the same portion of this food in order to feel full. If the caloric density is greater than about three, then the person will consume many calories if the person eats the same portion of this food in order to feel full. In general, the caloric density is about two if it is between 1.9 and 2.1 and the caloric density is about three if it is between 2.9 and 3.1. In other words, if a person consumes enough food to make them feel full and if they are eating low caloric density food, then they will be consuming fewer calories than if they are eating high caloric density foods.

Liquid food items with a caloric density of 0.4 or less are more desirable than those with a caloric density more than 0.4. This diet encourages the person to eat smaller portions of high caloric density foods because they are most likely to cause the person to gain body weight. The diet also encourages the person to eat larger portions of low caloric density foods because they are most likely to cause the person to lose body weight.

For example, chocolate pudding has a caloric density of about 1.3 because one half of a cup of chocolate pudding includes about 150 calories and weighs 113 grams (1.3=150 calories+113 grams). Since 1.3 is a relatively low caloric density number (i.e. it is less than two), one half of a cup of chocolate pudding is less likely to cause the person to gain weight. In contrast, a cheese Danish has a caloric density of about 3.9 because one cheese Danish includes about 353 calories and weighs about 91 grams (3.9=353 calories+91 grams). Since 3.9 is a high caloric density number (i.e. it is greater than three), a cheese Danish is more likely to cause the person to gain weight. Certain groups of foods, such as vegetables, fruits, fish, skinless chicken, etc., have low caloric densities and other groups of foods, such as candy, snack foods like potato chips, etc., all have high caloric densities.

It should be noted that there are some foods that have a relatively low caloric density and are very unhealthy. Consider for example an egg (the white and the yolk), which has a caloric density of 1.5. An egg will not cause the person to gain weight, but it weighs about 50 grams and includes about 1.6 grams of saturated fat. It has been well established that eating saturated fat leads to heart disease, so eggs are not a recommended food item in this diet unless the yolk is removed because the yolk includes almost all of the saturated fat. Egg whites have a caloric density of about 0.5 and contain very little, if any, saturated fat.

One reason this diet is effective is because a person feels fuller after consuming a smaller number of calories. It is important to understand what makes the person feel full and no longer hungry in order to understand how the diet works. While many factors go into what makes the person feel full and stop feeling hungry, the most important thing is the volume of the food the person is eating. The volume of the food is the size of the food and corresponds to its weight. This volume of food fills up the stomach and the intestines and stretches the walls of the stomach and intestines. As the stomach fills with food, the stomach distends, and the walls of the stomach stretch. When the walls of the stomach and intestines are stretched, the nerves in the walls of the stomach and intestines send signals to the brain which cause the person to feel full and to stop feeling hungry. Two solid foods of the same volume will make the person feel equally full and stop feeling hungry.

For example, if two and one half chocolate bars weigh 142 grams, then they weigh about as much as an average-sized apple. Because both the apple and the two and one half chocolate bars weigh the same amount, the person will basically feel equally full if the person eats either the apple or the two and one half chocolate bars. There is an important difference however. The apple has a low caloric density and the chocolate bars do not. The average-sized apple has about 81 calories, so its caloric density is about 0.6 (0.6=81 calories+142 grams). The two and a half chocolate bars have about 680 calories and a caloric density of about 4.8 (4.8=680 calories+142 grams).

Hence, whether the person eats an apple or two and a half chocolate bars, the person will feel about equally full since the apple weighs about as much as two and a half chocolate bars. However, if the person eats the apple, the person gains 81 calories and if the person eats two and a half chocolate bars, the person gains 680 calories. The person would have to eat about eight and a half apples to take in as many calories as there are in two and a half chocolate bars. An average size person would feel extremely full after eating that many apples, but it would be much easier to eat two and a half chocolate bars without feeling full. Hence, in general, if the person eats apples (in reasonable quantities) the person will lose weight and if the person eats chocolate bars the person will gain weight. Reasonable quantities are those that, when consumed, do not cause the person to gain weight undesirably.

It should be noted, however, that there are exceptions to the rule that foods of equal volume make the person feel equally full and not hungry. For example, a liquid food will not make the person feel as full as a solid food of the same volume. This is because solids move slower and liquids move faster through the stomach and intestines. In fact, for a meal of a given volume liquids pass through the stomach more quickly than solid foods do.

The emptying of liquids from the stomach in comparison to solids is even faster. Liquids empty at an exponential rate, while solids empty at a linear rate. In one example, half a liquid meal might be present in the stomach after 60 minutes, but by 90 minutes none of the liquid is left in the stomach. In contrast, three-quarters of a solid meal might be present after 60 minutes, one-half of the solid meal might be present after 120 minutes, one-quarter of the solid meal might be present after 180 minutes, and not until 240 minutes would almost none of the solid meal be present in the stomach. The liquid in this case makes the person feel full and stops the person from feeling hungry for 90 minutes, but the solid present in the stomach makes the person feel full and stops the person from feeling hungry for 240 minutes.

After food leaves the stomach it enters the first part of the small intestine, which is called the duodenum. Food present in the duodenum, whether liquid or solid, also stimulates nerves traveling to the brain that give the sensation of fullness. It is believed that solids stimulate the duodenum to produce a feeling of fullness for a longer period of time than do liquids. Foods with both a solid and a liquid component, such as most soups, are excellent for weight loss if the liquid component does not have many fats or dissolved sugars in it. Soups (except for cream-based soups) will fill the person up without giving the person many calories and will help the person lose weight.

In this embodiment, the diet has two stages, although it can have fewer or more stages. During a first stage, the person loses weight at a rate of one to two pounds a week until a desired weight is reached. Losing weight at a faster rate can be unhealthy in some instances. During a second stage, the person maintains the desired weight or a weight near the desired weight. The weight near the desired weight is typically within plus or minus five pounds of the desired weight, although it can be outside of this range. In both stages, it is preferred that the person eats three meals a day and eats healthy snacks two or three times a day. It will be understood by those skilled in the art that variations in the number of meals and snacks may occur, and while not preferred, as an example, four meals may be eaten per day. An example of a diet with one stage is when the person just wants to maintain his or her current weight. In this example, the person would not need to lose one to two pounds per week. An example of a diet with more than two stages is when the person is extremely obese. An extremely obese person may need to lose about fifty or more pounds. In these examples, the person may, preferably under medical supervision, lose more than two pounds per week before moving to a stage where he or she is losing one to two pounds per week. After this stage, the person can move to a stage where they are maintaining their weight.

The diet is typically begun by eating foods with a lower caloric density so that the person will lose weight. It is desired for the person to also eat foods that taste good so the person will stay with the diet. In stage one, the person should gradually lose weight until the desired weight is reached. Then the person should move into stage two and gradually expand what foods are eaten so that the person is consuming as many calories as the person burns and eating a wider variety of foods. At this point the person should maintain the desired weight or within an acceptable range of it.

The diet promotes the consumption of different types of foods since they have different effects on the body. Some foods are healthy and will help the person lose weight. However, there are also foods that will cause the person to lose weight but are unhealthy. There are even some foods, such as nuts, that will cause the person to gain weight but are so effective at preventing heart disease that the person should eat them anyway. Finally, there are many foods that cause the person to gain weight and are very unhealthy. Accordingly, there are a wide variety of food items which can be consumed for breakfast, lunch, dinner, and snacks, and a few are listed below. It should be noted, however, that there are other foods that are not listed here for simplicity and ease of discussion. These foods may also be acceptable for this diet if they have an acceptable caloric density and are eaten in portions which do not cause the person to consume more calories than are burned. Hence, the foods listed below can be replaced with one or more food items with the same or a similar caloric density.

For breakfast, the food items are generally selected from scrambled egg whites with low fat or non-fat cream cheese, cream of wheat, raspberries, blueberries, strawberries, bananas, an apricot-pineapple smoothie, oatmeal, raisin bran, Muesli cereal, French toast, waffles, and low fat pancakes. For lunch, the food items are generally selected from a turkey burger, pizza with non-fat mozzarella, chicken soft tacos with low-fat sour cream, tuna with mayo stuffed into tomato halves, curried chicken salad, nicoise salad with tuna and egg white, a roast beef or turkey sandwich, spaghetti with chunky tomato sauce and cubed chicken, spinach salad with grilled shrimp and papaya, salmon salad with cucumber and herbs, turkey chili with avocado, salad with crab cake and mango salsa, Japanese soba noodle salad, a grilled Portobello mushroom sandwich with roasted peppers, baby spinach, and pesto, turkey and black bean chili, Moroccan sliced chicken breast salad with olives, figs, and orange slices, spinach salad with roasted pear and sliced grilled turkey breast, salmon burger with baby spinach, tomato, and Dijon mustard on a pita, and sweet and sour chicken with vegetables.

For dinner, the food items are generally selected from braised veal chop, grilled sirloin steak with horseradish sauce, seared halibut kabobs, barbequed chicken breast (skinless of course), spaghetti with chunky tomato basil sauce, braised chicken breast, zucchini lasagna with non-fat mozzarella, pasta primavera, poached salmon with cucumber dill sauce, chicken vegetable stir fry, honey orange glazed salmon, grilled tuna kabob with mango salsa, eye of round roast, low-fat turkey meatloaf, potato gnocchi with marinara sauce, grilled turkey breast with roasted tomato salsa, herb crusted trout with lemon, roasted chicken breast with mango salsa, grilled shrimp skewer with pineapple and bell peppers, vegetable and turkey soft tacos with salsa and low fat or non-fat sour cream, grilled chicken skewers marinated in low fat or non-fat yogurt, cumin, lemon, and mint, baked red snapper with tomato, olives, and capers, baked eggplant with zucchini low fat or non-fat parmesan cheese, linguini with white clam sauce, and eye of round roast with roasted red pepper sauce.

For dessert, the food items are generally selected from a piece of blueberry, cherry or lemon meringue pie, jello, mixed berries with frozen yogurt, strawberry granita, a red wine poached pear, grilled fruit kabob with honeyed yogurt, watermelon, apple cinnamon crumble, a vanilla roasted plum, pineapple grilled with lemon and honey, citrus baked apple, chocolate, vanilla, or tapioca pudding with sliced banana, and angel food cake.

### Example Meal Plans

Included below are two different meal plans which are provided to illustrate the general idea of the diet. The first meal plan is a 1200 calorie a day meal plan for weight loss and the second meal plan is a 2000 calorie a day plan for weight maintenance. The 1200 calorie a day meal plan is for dieters who want to lose one to two pounds a week. If the dieter on this plan is losing less than one pound per week, then the dieter should eat less than 1200 calories per day. One way to do this is to decrease the portion size gradually. If the dieter is losing more than two pounds a week, then they are losing weight too quickly and the portion size should be increased gradually or a few foods with a higher caloric density should be added to the diet.

In a particular example, if a female starts off eating 1200 calories a day, but is losing three pounds per week (more than the goal range of one to two pounds per week), then it is recommended that she increase her caloric intake by adding higher caloric density foods and/or increasing their portion size to increase the number of calories she is consuming. Similarly, if she is losing less than one pound per week (less than the goal range of one to two pounds per week), then it is recommended that she decrease her caloric intake by adding lower caloric density foods and/or decreasing the portion size of the foods she is eating in order to decrease the number of calories she is consuming.

If she has reached her goal weight and wants to maintain it, then it is recommended that she start off consuming a fixed number of calories a day (e.g. 2000 calories a day for an average-sized woman), which is greater than the recommended number of calories she was consuming to lose weight (i.e. 1200 calories). If she is losing weight while trying to maintain a stable weight, then it is recommended that she increase her caloric intake by adding higher caloric density foods and/or increasing their portion size to increase the number of calories she is consuming. Similarly, if she is gaining weight while trying to maintain a stable weight, then it is recommended that she decrease her caloric intake by adding lower caloric density foods and/or decreasing the portion size of the foods she is eating in order to decrease the number of calories she is consuming.

It should be noted that the total number of calories for a person to lose one to two pounds a week or to maintain a desired weight depends on several factors. These factors can include the person's sex, age, size, metabolism, genetic history, level of activity, etc. Hence, for some people, 2000 calories will cause them to gain weight and for others it will cause them to lose weight. For many people, however, consuming 2000 calories per day will be the right number of calories and will not cause them to gain or lose weight. Accordingly, the number of calories shown here is for illustrative purposes and can be adjusted for the individual person. It should also be noted that it is typically discouraged for a normal-sized woman and man to consume fewer than 800 and 1000 calories per day, respectively.

These plans are meant to give the person an idea of the different foods one can eat in order to lose or maintain a desired body weight. Other foods can be substituted for those listed here as long as they have the same or a similar caloric density. An advantage of being able to substitute foods is that the dieter can adjust his or her diet to provide foods that he or she likes which provides a more varied diet. This increases the likelihood that the dieter will remain on the diet for a longer period of time.

For example, if the dieter is on Thursday afternoon of the 1200 calorie a day plan for weight loss, then the recommended afternoon snack is citrus baked apple with non-fat vanilla yogurt which includes about 140 calories. But if the dieter doesn't like yogurt and likes cottage cheese instead, then the dieter can have a cup of nonfat cottage cheese (123 calories) and ¼ of a cup of blueberries (20 calories) in place of the yogurt. The total number of calories for the day is about the same and these foods have a low caloric density.

Each of the meal plans below encourages the person to eat a certain number of times per day. In other words, consuming 1000 calories for breakfast and 200 calories for the rest of the day is discouraged. This is because the person is more likely to feel hungry. For example, the person eats only during meal times, such as breakfast, lunch, and dinner. The person is also allowed to eat two or three snacks a day so that the person will not feel hungry in between meals. One snack is between breakfast and lunch and another snack is between lunch and dinner. A third optional snack can be had after dinner if desired. It should be noted, however, that the number of times the person eats can be adjusted to the desires of an individual person. For example, some people may sleep and/or work non-traditional hours, so they may not be available for breakfast. Hence, the meal plans below are for illustrative purposes and other meal plans can be tailored for an individual's needs. It should be noted that the number of calories listed for each food are from the USDA and other sources may assign a different number of calories.

The 1200 Calorie a Day Plan for Weight Maintenance

Sunday:

Breakfast—Herbed scrambled egg substitute (½ cup, 53 calories) or egg white—scramble in 1 tablespoon non-fat cream cheese (15 calories) and chopped mixed herbs, a banana (109 calories), and a cup of coffee with skim milk and NutraSweet or another artificial sweetener.

Snack—White bean spread, 6 baby carrots (24 calories), Water.

Lunch—Turkey burger made with fat free turkey (193 calories), Placed on mixed greens and topped with tomato, sautéed onion, Dijon mustard, and non-fat mayonnaise, a sweet pickle (16 calories), and diet soda (4 calories).

Snack—Unsweetened apple sauce (½ cup, 53 calories).

Dinner—Butternut squash soup with 1 tablespoon low fat or fat-free sour cream and chopped chives (1 cup, 140 cal), braised veal chop with mushrooms and peppers (259 cal), 8 asparagus spears roasted with balsamic vinegar (28 calories), Water with lemon.

Snack—An orange (69 calories)

Monday:

Breakfast—Raisin bran cereal with skim milk (1 cup cereal with ½ cup milk, 229 calories), tomato juice (41 calories). If desired a cup of coffee with skim milk and flavored with NutraSweet or another artificial sweetener.

Snack—Blueberries (½ cup, 41 calories).

Lunch—Turkey sandwich—add arugula, roasted red peppers, and low-fat pesto mayo; use lower calorie bread, apple coleslaw (½ cup, 42 calories), diet soda (4 calories).

Snack—Smoothie-1 banana, ½ cup non-fat yogurt, and 2 strawberries.

Dinner—Seared halibut kabobs with lemon, Italian vegetable stew (150 calories), ½ cup brown rice (108 calories), water with cucumber slices.

Snack—Low calorie jello.

Tuesday:

Breakfast—Low-fat French toast (2 slices, 252 calories) with blueberries (½ cup, 41 calories), two vegetarian breakfast links (63 calories). If desired a cup of coffee with non-fat skim milk and flavored with NutraSweet or another artificial sweetener.

Snack—Half an apple sliced (41 calories) drizzled with 2 tablespoons non-fat vanilla yogurt.

Lunch—A can of light tuna, with mixed in fat-free mayonnaise, chopped scallion, dill, celery, and lemon juice, stuffed into 2 tomato halves (410 calories), water or diet soda.

Snack—10 frozen grapes (36 calories).

Dinner—Minestrone soup (82 calories), zucchini pasta “lasagna” with non-fat mozzarella, green beans with balsamic vinegar (110 calories), water with lemon.

Snack—Mixed berries (1 cup, 60 calories) with ½ cup non-fat frozen yogurt (50 calories).

Wednesday

Breakfast—Muesli cereal “parfait”-Muesli (⅔ cup, 190 calories) layered with low-fat yogurt and 4 sliced strawberries (16 cal). If desired a cup of coffee with skim milk and flavored with NutraSweet or another artificial sweetener.

Snack—Honeydew melon (⅛ melon, 56 calories) with non-fat cottage cheese (¼ cup, 31 cal).

Lunch—Curried chicken salad with grapes in lettuce wrap, water or diet soda.

Snack—Eggplant dip (½ cup, 40 calories) with celery sticks (1 stalk, 6 calories).

Dinner—Chopped vegetable salad (100 calories), fettuccine with roasted vegetables (225 calories), water.

Snack—Strawberry granita.

Thursday:

Breakfast—2 low-fat waffles (166 calories), a glass of skim milk (86 calories). If desired a cup of coffee with skim milk and flavored with NutraSweet or another artificial sweetener.

Snack—Roasted artichoke with Dijon dipping sauce (non-fat mayo, Dijon mustard, and lemon juice) (57 calories).

Lunch—Nicoise salad—3 ounces canned light tuna in water atop mixed greens, green beans, 1 sliced baby potato, red onion, black olives, 2 cherry tomatoes, and light vinaigrette (293 calories), water or diet soda.

Snack—Citrus baked apple with non-fat vanilla yogurt (140 calories).

Dinner—1 cup Gazpacho (28 calories), poached salmon (6 ounces, 368 calories) with non-fat cucumber dill yogurt sauce, sautéed carrot and zucchini strips.

Snack—Fat-free chocolate pudding (107 calories).

Friday:

Breakfast—Oatmeal (1 cup, 145 calories) with cinnamon and 1 teaspoon drizzle maple syrup, ½ cup mixed berries (30 calories). If desired a cup of coffee with skim milk and flavored with NutraSweet.

Snack—4 tablespoons of hummus (92 calories) and 8 baby carrots (32 calories).

Lunch—Sliced turkey lettuce wrap with 1 sliced avocado, tomato, and fat-free mayonnaise (140 calories), diet soda (4 calories).

Snack—An orange (62 calories).

Dinner—Chicken and vegetable stir-fry, ½ cup brown rice (108 calories), water.

Snack—Pumpkin pie (1 piece, 229 calories).

Saturday:

Breakfast

2 pancakes prepared from lower calorie mix (148 calories): add ½ cup blueberries to mix (41 cal). If desired a cup of coffee with skim milk and flavored with NutraSweet or another artificial sweetener.

Snack—Banana smoothie: blend 1 cup orange juice, 1 cup vanilla low fat yogurt, and a banana (206 calories).

Lunch—Roast beef sandwich (346 calories) with grilled red onion, Dijon mustard, and horseradish sauce, a sweet pickle (18 calories), sugar-free lemonade (5 calories).

Snack—A peach (42 calories).

Dinner—Orange and cumin glazed salmon with ½ cup toasted couscous (431 calories), sesame broccoli and carrots (73 calories), water with lemon.

Snack—Red wine poached pear (150 calories).

The 2000 Calorie a Day Plan for Weight Maintenance

Sunday:

Breakfast—Scrambled egg substitute (½ cup) or egg white: scramble in 2 pieces of fat free American cheese (168 calories)—top with salsa and 2 slices avocado; orange banana smoothie: blend 1 banana, an 8 ounce glass of orange juice, and 1 cup vanilla low-fat yogurt (206 calories). If desired, a cup of coffee with skim milk and flavored with NutraSweet or another artificial sweetener.

Snack—Hummus (6 tablespoons, 138 calories), tahini (1 tablespoon, 89 calories), ½ piece of whole wheat pita (83 calories), 5 black olives (25 calories), water with lemon.

Lunch—Turkey burger made with fat free turkey (193 calories) Place on mixed greens and top with tomato, sautéed mushrooms, onions, and Dijon mustard. A sweet pickle (16 calories), diet soda (4 calories).

Snack—12 macadamia nuts (203 calories).

Dinner—Butternut squash soup (1 cup, 78 calories), braised veal cutlet with mushrooms and peppers (6 ounces, 358 calories), mashed potatoes, preferably made with water or skim milk, no butter or margarine. (¾ cup, 122 calories), spinach sauté, a glass of red wine (3.5 ounces, 74 calories), water.

Snack—Non-fat vanilla pudding (½ cup, 105 calories).

Monday:

Breakfast—Raisin bran cereal with skim milk (1 cup cereal with ½ cup milk, 229 calories), blueberries (½ cup, 41 calories). If desired a cup of coffee with skim milk and flavored with NutraSweet or another artificial sweetener.

Snack—Non-fat yogurt with fruit (8 ounces, 213 calories).

Lunch—Turkey wrap: add lettuce, tomatoes, roasted red pepper, onions, low fat mayonnaise, preferably lower calorie whole wheat tortilla bread, apple coleslaw, an orange (62 calories), diet soda (4 calories).

Snack—Grilled artichoke with dipping sauce (non-fat mayonnaise, Dijon mustard, and lemon juice), water.

Dinner—Grilled tuna kabob with mango salsa, rice, zucchini “pasta” with 1 tablespoon olive oil (70 calories), a glass of white wine if desired (3.5 ounces, 70 calories), water.

Snack—Apple cinnamon crumble (356 calories).

Tuesday

Breakfast—Low fat French toast (2 slices, 252 calories), reduced calorie pancake syrup (2 tablespoons, 50 calories), ½ cup blueberries (41 calories), 2 vegetarian breakfast links (63 calories), a glass of orange juice (8 ounces, 112 calories). If desired a cup of coffee with skim milk and flavored with NutraSweet or another artificial sweetener.

Snack—Half an apple sliced (41 calories), natural peanut butter (2 tablespoons, 188 calories).

Lunch—A can of light tuna with mixed in fat-free mayonnaise chopped scallion, dill, celery, and lemon juice stuffed into 2 tomato halves, water or diet soda.

Snack—Unsweetened apple sauce (½ cup, 53 calories).

Dinner—Vegetable soup (130 calories), skinless grilled chicken breast with barbeque sauce (308 calories) and ½ grilled peach, potato salad, carrot jicama salad, a glass of red wine (74 calories), water.

Snack—Mixed berries (1 cup, 60 calories) with non-fat sour cream (2 tablespoons, 24 calories).

Wednesday:

Breakfast—Muesli cereal “parfait” (⅔ cup, 190 calories) with non-fat vanilla yogurt (½ cup, 44 calories) and sliced banana. If desired a cup of coffee with skim milk and flavored with NutraSweet.

Snack—Honeydew melon with fat-free cottage cheese (⅛ melon, 56 calories and ½ cup cottage cheese, 62 calories).

Lunch—Curried chicken salad with grapes, scallion, non-fat mayonnaise in lettuce wrap, and water or diet soda.

Snack—Fat-free chocolate pudding (4 ounces, 107 calories) with sliced toasted almonds.

Dinner—Vegetable antipasto (66 calories), spaghetti with chunky tomato basil sauce and roasted veggies, a glass of Chianti if desired (74 calories), water.

Snack—A piece of angel food cake (72 calories).

Thursday:

Breakfast—1 slice toasted low calorie bread with one tablespoon natural peanut butter, 1 tablespoon low calorie jam (116 calories), and ½ sliced banana, a glass of orange juice (112 calories), a glass of skim milk (86 calories). If desired a cup of coffee with skim milk and flavored with NutraSweet.

Snack—Vanilla non-fat yogurt with ½ chopped apple and raisins.

Lunch—Turkey/black bean chili with 1 tablespoon shredded non-fat cheese, an apple (81 calories), water or diet soda.

Snack—Fat-free chocolate pudding (107 calories) with 1 tablespoon sliced almonds.

Dinner—Lentil soup (80 calories), baby spinach topped with grilled salmon, cucumber, tomato, and dill dressing (247 calories), a glass of red wine (3.5 oz, 70 calories).

Snack—A piece of cherry pie (304 calories).

Friday:

Breakfast—Oatmeal (1 cup, 145 calories) with a sliced banana (109 calories) and cinnamon, apricot-pineapple smoothie (225 calories), two vegetarian breakfast links (63 calories). If desired a cup of coffee with skim milk and flavored with NutraSweet or another artificial sweetener.

Snack—four tablespoons of hummus (92 calories) with asparagus spears (28 calories) and 8 baby carrots (32 calories)

Lunch—Sliced turkey sandwich on low calorie bread with tomatoes, cranberry sauce, and fat-free mayonnaise (320 calories), diet soda (4 calories).

Snack—Roasted corn on the cob (90 cal).

Dinner—Tomato basil soup (120 calories), braised skinless (grilled) chicken breast (284 calories) with artichoke hearts (120 calories) and tomato (26 calories), a glass (3.5 oz) of red wine (74 calories), water.

Snack—Light yellow cake (1 piece, 181 calories).

Saturday:

Breakfast—2 pancakes prepared from lower calorie mix (148 calories) with a mashed banana (109 cal) and cinnamon added to mixture, low calorie syrup (2 tablespoons, 50 calories). If desired a cup of coffee with skim milk and flavored with NutraSweet.

Snack—12 macadamia nuts (203 calories).

Lunch—Roast beef sandwich (346 calories) with horseradish Dijon sauce and grilled onions, a sweet pickle (16 calories), potato salad (½ cup, 179 calories), sugar-free lemonade (5 calories).

Snack—A peach (42 calories).

Dinner—Sashimi appetizer (2 pieces, 164 calories), honey orange glazed salmon, stir fried vegetables, a glass of red wine (3.5 ounces, 74 calories), iced green tea.

Snack—Roasted plums.

Thus, a diet is disclosed which allows a person to lose weight and/or maintain a desired body weight in a healthy manner. The person on this diet can choose food items based on their caloric density. The diet encourages the consumption of solid food items with a caloric density of less than about two and the limitation of the consumption of food items with a caloric density over about three. The lower the caloric density, the more it will promote weight loss while still making the person feel full. It will also make it easier to maintain a desired body weight. The diet encourages the consumption of larger portions of liquid food items with a caloric density of 0.4 or less and the consumption of smaller portions of liquids with a caloric density more than 0.4. The diet provides a wide variety of foods which encourages the person to remain on the diet. This is because the person can choose food items which he or she likes.

The present invention is described above with reference to preferred embodiments. However, those skilled in the art will recognize that changes and modifications may be made in the described embodiments without departing from the nature and scope of the present invention. Various further changes and modifications will readily occur to those skilled in the art. To the extent that such modifications and variations do not depart from the spirit of the invention, they are intended to be included within the scope thereof.

Having fully described the invention in such clear and concise terms as to enable those skilled in the art to understand and practice the same, the invention claimed is:

1. A method of dieting, comprising:
providing a food item;
determining the caloric density of the food item; and
consuming an amount of the food item based on its caloric density.

2. The method of claim 1, further including consuming a larger amount of the food item if its caloric density is less than or equal to about two.

3. The method of claim 1, further including decreasing a portion of the food item if its caloric density is greater than about two.

4. The method of claim 1, further including determining the number of calories of the food item and at least one of its weight and volume.

5. The method of claim 1, wherein the food item is provided because it is healthy.

6. A method of dieting, comprising:
providing a first food item;
determining the caloric density and the number of calories of the first food item;
providing a second food item;
determining the caloric density and the number of calories of the second food item;
adjusting amounts of the first and second food items based on a difference between the caloric densities between them; and
consuming the amounts of the first and second food items, the number of calories in the first and second food items being less than or equal to a predetermined amount of calories.

7. The method of claim 6, further including reducing the first amount of the first food item if its caloric density is greater than a first predetermined value.

8. The method of claim 7, wherein the first predetermined value is about two.

9. The method of claim 6, wherein the first food item has a caloric density greater than the caloric density of the second food item and the amount of the first food item is less than the amount of the second food item.

10. The method of claim 6, wherein the predetermined amount of calories is chosen so that a person on the diet loses body weight at a desired rate.

11. The method of claim 6, wherein the predetermined amount of calories is chosen so that a person on the diet maintains a desired body weight.

12. The method of claim 6, wherein the step of determining the caloric density of the first food item includes dividing the number of calories in the first food item by its weight or volume.

13. A method of dieting, comprising:
determining a desired number of calories to consume;
providing a first portion of food with a first caloric density and a first number of calories;
providing a second portion of food with a second caloric density and a second number of calories, the combination of the first and second number of calories being less than or equal to the desired number of calories and the first caloric density being less than the second caloric density; and
adjusting the first and second portions of food so that the first portion of food is greater than the second portion of food.

14. The method of claim 13, wherein the desired number of calories consumed reduces a person's body weight by about one to two pounds per week.

15. The method of claim 13, wherein the desired number of calories consumed maintains a person's body weight.

16. The method of claim 13, wherein the first portion of food is a first food item with a caloric density less than or equal to about two.

17. The method of claim 13, wherein the second portion of food is a second food item with a caloric density greater than about two.

18. The method of claim 13, wherein the first portion of food includes a liquid with a caloric density less than or equal to 0.4.

19. The method of claim 13, further including determining the first and second number of calories from one or more dietary guidelines.

20. The method of claim 13, further including determining the caloric density of the first food item by dividing its number of calories by its weight or volume.
